(19) United States
(12) Patent Application Publication
(10) Pub. No.: US 2025/0308674 A1
(43) Pub. Date: Oct. 2, 2025

(54) IMAGE PROCESSING APPARATUS, IMAGE PROCESSING METHOD, IMAGE PROCESSING PROGRAM, LEARNING DEVICE, LEARNING METHOD, AND LEARNING PROGRAM

(71) Applicant: FUJIFILM CORPORATION, Tokyo (JP)

(72) Inventor: Akira KUDO, Tokyo (JP)

(21) Appl. No.: 19/089,004

(22) Filed: Mar. 25, 2025

(30) Foreign Application Priority Data
Mar. 26, 2024 (JP) 2024-049673

(51) Int. Cl. G16H 30/40; G06V 10/26; G06V 10/77; G06V 10/82

(52) U.S. Cl. CPC G16H 30/40; G06V 10/26; G06V 10/7715; G06V 10/82

(57) ABSTRACT

There are provided an image processing apparatus, an image processing method, an image processing program, a learning device, a learning method, a learning program, and a derivation model capable of performing domain conversion of an image in which an anatomical structure is maintained. A processor derives a second image from a first image of a first modality, and derives a third image of a second modality different from the first modality, from the second image.

### CROSS REFERENCE TO RELATED APPLICATIONS

The present application claims priority from Japanese Patent Application No. 2024-049673, filed on Mar. 26, 2024, the entire disclosure of which is incorporated herein by reference.

### BACKGROUND

### Technical Field

The present disclosure relates to an image processing apparatus, an image processing method, an image processing program, a learning device, a learning method, and a learning program.

### Related Art

In a medical field, advances in various modalities (that is, imaging apparatuses or imaging methods), such as a computed tomography (CT) apparatus, a magnetic resonance imaging (MRI) apparatus, an ultrasound imaging apparatus, a positron emission tomography (PET) apparatus, and an X-ray imaging apparatus, have made it possible to perform image diagnosis using a medical image with higher quality. Three-dimensional images acquired by such modalities have different image characteristics depending on the modality. Therefore, in clinical practice, there is a case in which diagnosis is performed by comparing images of a plurality of modalities. For example, in a vertebral body examination, an MRI image acquired by an MRI apparatus is used for a soft tissue, and a CT image acquired by a CT apparatus is used for a bone structure.

On the other hand, a computer aided diagnosis (computer aided diagnosis, computer aided detection: CAD) system using artificial intelligence (AI) is generally constructed for each modality that captures a target medical image. Here, in a case in which medical images are of different modalities, such as a CT apparatus and an MRI apparatus, an image expression format differs. For example, even in a case in which a human tissue included in the images is the same, a density is different between a CT image and an MRI image. In addition, the MRI images have various imaging conditions such as a T1-weighted image, a T2-weighted image, a fat-suppressed image, and a diffusion-weighted image, and a magnetic field intensity and the like vary depending on the imaging conditions, so that an appearance of the generated image, that is, an image expression format is different for each image. For example, in the T1-weighted image, an adipose tissue appears primarily white, water, a liquid component, and a cyst appear black, and a tumor appears slightly black. In addition, in the T2-weighted image, not only the adipose tissue but also water, a liquid component, and a cyst appear white. In addition, the MRI images may have different expression formats depending on a manufacturer of the MRI apparatus even under the same imaging conditions. As described above, even in a case of the images of the same modality, the expression formats are different due to different imaging conditions and the like.

Therefore, in a case in which the CAD system is applied to an image in an expression format different from the image in the expression format used for learning, the accuracy of image analysis may be reduced. Therefore, for example, there is a demand for a high-performance image converter that performs domain conversion of images between different modalities or between images having different expression formats, such as processing of generating a pseudo MRI image from a CT image, or conversely, processing of generating a pseudo CT image from an MRI image.

In order to respond to such a demand, various methods of generating images of different domains using a generative adversarial network (GAN) are proposed. For example, in Jun-Yan Zhu, et al., Unpaired Image-to-Image Translation using Cycle-Consistent Adversarial Networks, Berkeley AI Research (BAIR) laboratory, UC Berkeley, ArXiv: 1703.10593, 24 Aug. 2020, a method of converting images of two different domains into each other through adversarial learning is proposed. In addition, in Yunjey Choi, et al., StarGAN: Unified Generative Adversarial Networks for Multi-Domain Image-to-Image Translation, arXiv:1711.09020, 21 Sep. 2018, a method of mutually converting images of two or more different domains into each other through adversarial learning is proposed.

As described above, there are a plurality of types of MRI images having different expression formats. Therefore, in a case in which a CT image is converted into an MRI image, there is a possibility that an anatomical structure included in the CT image cannot be maintained in the MRI image depending on the expression format of the MRI image.

### SUMMARY OF THE INVENTION

The present disclosure has been made in view of the above circumstances, and an object of the present disclosure is to enable domain conversion of an image in which an anatomical structure is maintained.

According to the present disclosure, there is provided an image processing apparatus comprising: a processor, in which the processor derives a second image from a first image of a first modality, and derives a third image of a second modality different from the first modality, from the second image.

According to the present disclosure, there is provided a learning device that performs learning for constructing a segmentation model for segmenting an anatomical structure included in an image of a second modality, the learning device comprising: a processor, in which the processor performs the learning using the third image derived by the image processing apparatus according to the present disclosure as learning data.

According to the present disclosure, there is provided an image processing method comprising: causing a computer to execute deriving a second image from a first image of a first modality, and deriving a third image of a second modality different from the first modality, from the second image.

According to the present disclosure, there is provided a learning method for performing learning for constructing a segmentation model for segmenting an anatomical structure included in an image of a second modality via a computer, in which the learning is performed using the third image derived by the image processing apparatus according to the present disclosure as learning data.

According to the present disclosure, there is provided an image processing program causing a computer to execute: a procedure of deriving a second image from a first image of a first modality; and a procedure of deriving a third image of a second modality different from the first modality, from the second image.

According to the present disclosure, there is provided a learning program causing a computer to execute learning for constructing a segmentation model for segmenting an anatomical structure included in an image of a second modality, the learning program causing the computer to execute the learning using the third image derived by the image processing apparatus according to the present disclosure as learning data.

According to the present disclosure, it is possible to perform domain conversion of an image in which an anatomical structure is maintained.

### BRIEF DESCRIPTION OF THE DRAWINGS

FIG. 1 is a hardware configuration diagram showing an outline of a diagnosis support system to which an image processing apparatus and a learning device according to an embodiment of the present disclosure are applied.

FIG. 2 is a diagram showing a hardware configuration of the image processing apparatus according to the present embodiment.

FIG. 3 is a diagram showing a functional configuration of the image processing apparatus according to the present embodiment.

FIG. 4 is a diagram for describing a conversion model.

FIG. 5 is a diagram showing a mask.

FIG. 6 is a diagram for describing learning for constructing a first conversion model.

FIG. 7 is a diagram for describing learning for constructing a second conversion model.

FIG. 8 is a diagram for describing learning for constructing a conversion model and a segmentation model.

FIG. 9 is a diagram showing a display screen of a third image.

FIG. 10 is a flowchart showing processing performed by the image processing apparatus according to the present embodiment.

FIG. 11 is a diagram showing a hardware configuration of the learning device according to the present embodiment.

FIG. 12 is a diagram showing a functional configuration of the learning device according to the present embodiment.

FIG. 13 is a diagram for describing training of a segmenter for constructing a segmentation model.

FIG. 14 is a flowchart showing processing performed by the learning device according to the present embodiment.

### DETAILED DESCRIPTION

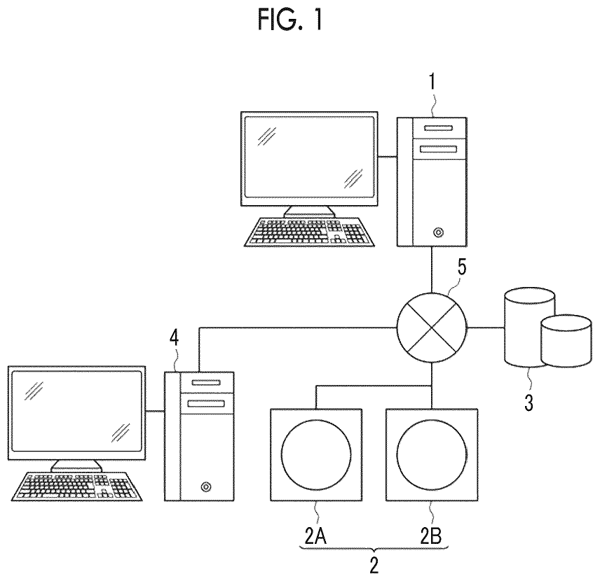

Hereinafter, an embodiment of the present disclosure will be described with reference to the drawings. FIG. 1 is a hardware configuration diagram showing an outline of a diagnosis support system to which an image processing apparatus and a learning device according to an embodiment of the present disclosure are applied. As shown in FIG. 1, in the diagnosis support system, an image processing apparatus 1, a modality 2, an image storage server 3, and a learning device 4 according to the present embodiment are connected to each other in a communicable state via a network 5.

The modality 2 is an apparatus that generates an image representing a diagnosis target part of a subject by imaging the part, and specifically, is a CT apparatus, an MRI apparatus, an ultrasound imaging apparatus, a PET apparatus, an X-ray imaging apparatus, and the like. The image of the subject generated by the modality 2 is transmitted to the image storage server 3 and stored therein. In the present embodiment, it is assumed that the modality 2 includes a CT apparatus 2A and an MRI apparatus 2B.

In the present embodiment, a three-dimensional image and a two-dimensional image are acquired by the CT apparatus 2A or the MRI apparatus 2B. In the present embodiment, both the three-dimensional image and the two-dimensional image include a tomographic image, but the three-dimensional image includes either or both of a plurality of tomographic images in which at least one of a slice interval or a slice thickness is smaller than that of the two-dimensional image and an image generated from the plurality of tomographic images in which each pixel is represented by three-dimensional coordinates. For example, the three-dimensional image includes a plurality of tomographic images in which at least one of a slice thickness or a slice interval is 5 mm or less. The three-dimensional image is acquired by the CT apparatus 2A or the MRI apparatus 2B performing three-dimensional imaging on the subject.

Meanwhile, the two-dimensional image is acquired by the CT apparatus 2A or the MRI apparatus 2B performing two-dimensional imaging on the subject. The two-dimensional image includes either or both of a plurality of tomographic images in which a slice interval is larger than that of the tomographic images included in the three-dimensional image and at least one or more tomographic images in which a slice thickness is larger than that of the tomographic images included in the three-dimensional image. The tomographic images include an image in which each pixel is represented by two-dimensional coordinates. In a case in which the three-dimensional image or the two-dimensional image is composed of a plurality of tomographic images, the tomographic images include position coordinates of each tomographic image in an imaging direction. Therefore, in the entire plurality of tomographic images, each pixel is represented by three-dimensional coordinates. The imaging direction is, for example, a direction perpendicular to a tomographic plane represented by the tomographic image.

The image storage server 3 is a computer that stores and manages various data, and comprises a large-capacity external storage device and database management software. The image storage server 3 communicates with another device via the wired or wireless network 5 and transmits and receives image data and the like. Specifically, the image storage server 3 acquires various data including image data of the image generated by the modality 2 via the network, and stores and manages the various data in a recording medium such as the large-capacity external storage device. A storage format of the image data and the communication between the respective devices via the network 5 are based on a protocol such as digital imaging and communication in medicine (DICOM). In addition, in the present embodiment, the image storage server 3 also stores and manages learning data described below.

The image processing apparatus 1 and the learning device 4 according to the present embodiment are computers in which an image processing program and a learning program according to the present embodiment are respectively installed. The computer may be a workstation or a personal computer directly operated by a doctor performing diagnosis, or may be a server computer connected to them via a network. The image processing program and the learning program are stored in a storage apparatus of a server computer connected to the network or in a network storage in a state where the network storage can be accessed from an outside, and are downloaded to and installed on a computer used by a doctor upon request. Alternatively, the image processing program and the learning program are distributed by being recorded on a recording medium such as a digital versatile disc (DVD) or a compact disc read only memory (CD-ROM) and are installed on the computer from the recording medium.

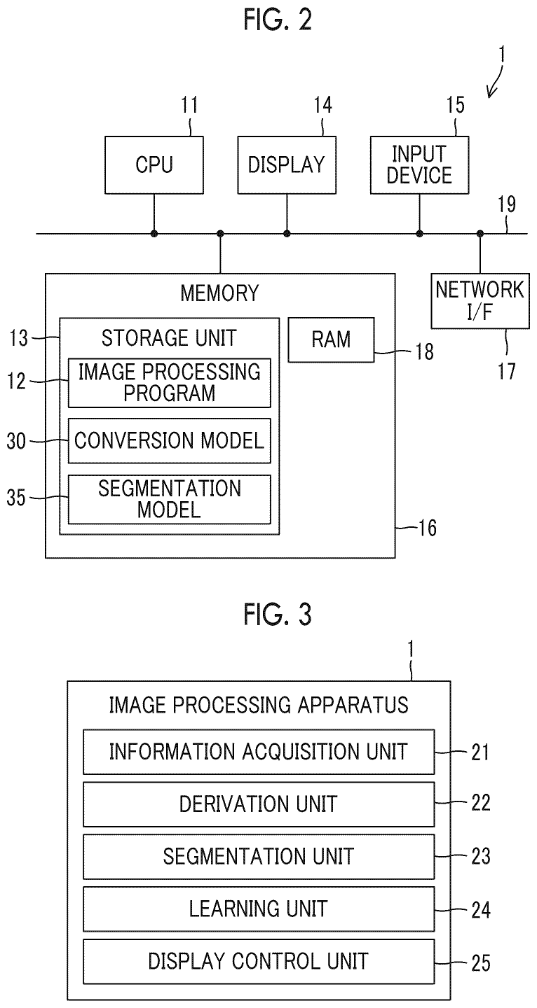

Hereinafter, the image processing apparatus according to the present embodiment will be described. FIG. 2 is a diagram showing a hardware configuration of the image processing apparatus according to the present embodiment. As shown in FIG. 2, the image processing apparatus 1 includes a central processing unit (CPU) 11, a display 14, an input device 15, a memory 16, and a network interface (I/F) 17 connected to the network 5. The CPU 11, the display 14, the input device 15, the memory 16, and the network I/F 17 are connected to a bus 19. The CPU 11 is an example of a processor in the present disclosure.

The memory 16 includes the storage unit 13 and a random access memory (RAM) 18. The RAM 18 is a memory for primary storage and is, for example, a RAM such as a static random access memory (SRAM) or a dynamic random access memory (DRAM).

The storage unit 13 is a non-volatile memory, and is implemented by at least one of, for example, a hard disk drive (HDD), a solid state drive (SSD), an electrically erasable and programmable read only memory (EEPROM), or a flash memory. An image processing program 12 according to the present embodiment is stored in the storage unit 13 as a storage medium. The CPU 11 reads out the image processing program 12 from the storage unit 13, loads the read-out image processing program 12 into the RAM 18, and executes the loaded image processing program 12. The storage unit 13 also stores a conversion model 30 and a segmentation model 35, which will be described below.

The display 14 is a device that displays various screens and is, for example, a liquid crystal display or an electro luminescence (EL) display. The input device 15 is a device for a user to provide input and is, for example, at least any of a keyboard, a mouse, a microphone for voice input, a touchpad for proximity input including a contact, or a camera for gesture input. The network I/F 17 is an interface for connecting to the network 5.

Next, a functional configuration of the image processing apparatus according to the present embodiment will be described. FIG. 3 is a diagram showing the functional configuration of the image processing apparatus according to the present embodiment. As shown in FIG. 3, the image processing apparatus 1 comprises an information acquisition unit 21, a derivation unit 22, a segmentation unit 23, a learning unit 24, and a display control unit 25. In a case in which the CPU 11 executes the image processing program 12, the CPU 11 functions as the information acquisition unit 21, the derivation unit 22, the segmentation unit 23, the learning unit 24, and the display control unit 25.

The information acquisition unit 21 acquires a first image G1 to be processed from the image storage server 3 via the network 5. In the present embodiment, it is assumed that a three-dimensional CT image including a plurality of tomographic images having a small slice thickness is acquired as the first image G1. The CT apparatus 2A is an example of a first modality of the present disclosure, and the first image G1, which is a three-dimensional CT image, is an example of a first image of the first modality of the present disclosure. In the present embodiment, the image of the modality is, for example, an image expressed by an imaging method of the modality. For example, the CT image is an image expressed by an imaging method of the CT apparatus. The image expressed by the imaging method includes at least one of an actual image that is actually captured by the CT apparatus or a pseudo image that is derived in a pseudo manner by image processing.

The derivation unit 22 derives a third image G3 of a second modality different from the first modality, from the first image G1 of the first modality using a conversion model.

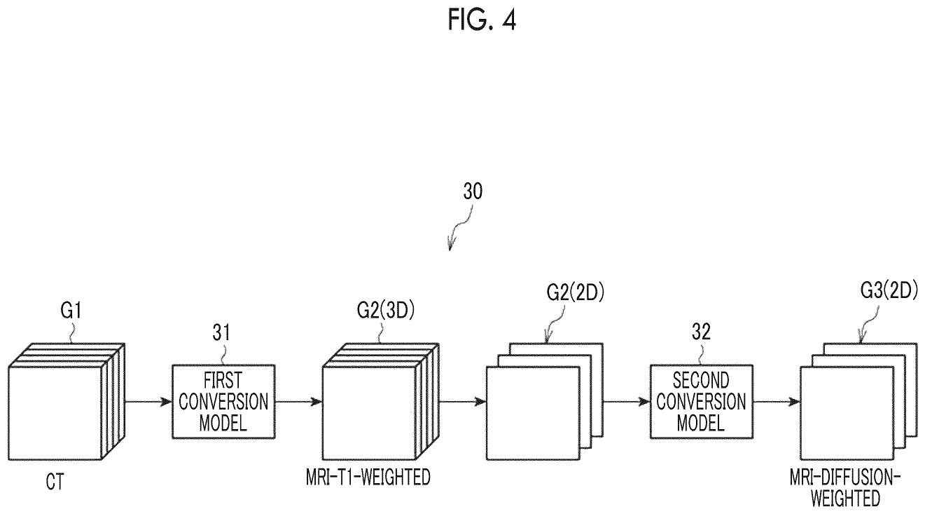

FIG. 4 is a schematic block diagram showing a configuration of the conversion model. As shown in FIG. 4, the conversion model 30 includes a first conversion model 31 and a second conversion model 32. The derivation unit 22 derives a second image G2 from the first image G1 of the first modality, that is, from the three-dimensional CT image using the first conversion model 31, and derives the third image G3 of the second modality different from the first modality, that is, an MRI image from the second image G2 using the second conversion model 32. The MRI apparatus 2B is an example of a second modality of the present disclosure, and the third image G3, which is an MRI image, is an example of a third image of the second modality of the present disclosure.

The first conversion model 31 is, for example, a three-dimensional convolutional neural network (3D-CNN), and derives the second image G2 in an expression format different from the expression format of the input first image G1 by performing convolution on the three-dimensional image using a three-dimensional filter. In the present embodiment, the first conversion model 31 is a domain conversion network for converting the expression format of the input three-dimensional CT image into an expression format of a three-dimensional MRI image, that is, for converting the domain.

The first conversion model 31 converts the three-dimensional CT image, which is the first image G1, to derive the three-dimensional MRI image that can three-dimensionally express the anatomical structure included in the three-dimensional CT image as the second image G2. Examples of the MRI image derived by the first conversion model 31 include a T1-weighted image or a T2-weighted image having high general-purpose properties. In the present embodiment, it is assumed that the T1-weighted image is derived as the second image G2. In the following description, the T1-weighted image and the T2-weighted image may be referred to as an MRI-T1-weighted image and an MRI-T2-weighted image, respectively.

The second conversion model 32 is, for example, a two-dimensional convolutional neural network (2D-CNN), and is a domain conversion network that derives the third image G3 in an expression format different from the expression format of the input second image G2 by performing convolution on an image represented by two-dimensional coordinates using a two-dimensional filter. Since the second conversion model 32 is a 2D-CNN, at least one tomographic image is extracted from the three-dimensional second image G2 derived by the first conversion model 31, and the second image G2, which is the extracted tomographic image, is input to the second conversion model 32. In the present embodiment, since the MRI image derived by the first conversion model 31 is the T1-weighted image, the extracted tomographic image is the T1-weighted image represented by the two-dimensional coordinates. In a case in which a plurality of tomographic images having a slice interval larger than that of the tomographic image included in the three-dimensional second image G2 are extracted, or in a case in which one or more tomographic images having a larger slice thickness are extracted, the plurality of extracted tomographic images or one or more extracted tomographic images constitute the two-dimensional image.

The second conversion model 32 converts the expression format of the T1-weighted image, which is the input tomographic image represented by the two-dimensional coordinates, into a tomographic image in an expression format different from the T1-weighted image, such as the T2-weighted image, a diffusion-weighted image, a fat-suppressed image, and a FLAIR image. In addition, an appearance of the MRI image varies depending on a manufacturer that manufactures the apparatus. Therefore, the second conversion model 32 may convert the expression format of the T1-weighted image, which is a tomographic image, into a tomographic image in an expression format that corresponds to the appearance of the MRI image from various manufacturers. In FIG. 4, the second conversion model 32 converts the T1-weighted image into the diffusion-weighted image.

In the present embodiment, the second conversion model 32 may be constructed to convert the T1-weighted image into an MRI image in one type of expression format other than the T1-weighted image, or may be capable of converting the T1-weighted image into MRI images in a plurality of types of expression formats. In the latter case, in addition to the T1-weighted image, information for designating the expression format to be converted is input to the second conversion model 32, and the input T1-weighted image is converted into an MRI image in the input expression format. In the following description, it is assumed that the second conversion model 32 converts the input T1-weighted image into an MRI image in one type of expression format.

The second conversion model 32 converts the second image G2, which is an image represented by two-dimensional coordinates, into the third image G3, which is an image represented by two-dimensional coordinates in a different expression format from the second image G2. Here, the second conversion model 32 performs processing of converting one tomographic image, which is formed by two-dimensional coordinates, included in the second image G2 into one tomographic image, which is formed by two-dimensional coordinates, included in the third image G3. In a case in which the second image G2 includes a plurality of tomographic images, the second conversion model 32 converts each of the plurality of tomographic images into the third image G3 including a plurality of tomographic images. Here, in the conversion processing performed by the second conversion model 32, the coordinates indicated by the tomographic image included in the second image G2 to be input and the tomographic image included in the third image G3 to be output are the same. Therefore, in a case in which at least one of the slice interval of the plurality of tomographic images or the slice thickness of at least one tomographic image input from the second image G2 to the second conversion model 32 is small, at least one of the slice interval of the plurality of tomographic images or the slice thickness of at least one tomographic image included in the third image G3 derived by the second conversion model 32 is also small. Therefore, the tomographic images are extracted from the three-dimensional second image G2 based on at least one of the number of slices, the slice interval, or the slice thickness according to the purpose of use, and are converted into the third image G3.

For example, in a case in which the three-dimensional second image G2 is composed of a plurality of tomographic images having a first slice interval, the three-dimensional third image G3 consisting of the plurality of tomographic images having the first slice interval can be acquired by converting each of the plurality of tomographic images having the first slice interval included in the three-dimensional second image G2 using the second conversion model 32. Meanwhile, scout images used for positioning at the time of imaging have a slice interval of, for example, 7 mm or more, and only a few of them are acquired. Therefore, the third image G3 consisting of tomographic images with the slice interval and the number that can be used as the scout image can be acquired by extracting the plurality of tomographic images from the second image G2 at the slice interval of 7 mm or more and converting the extracted tomographic images using the second conversion model 32.

The third image G3 derived as described above is not an actual image actually captured by the MRI imaging apparatus, but is a pseudo image that is derived in a pseudo manner. Therefore, the derivation unit 22 may assign information indicating that the third image G3 is a pseudo image, to the third image G3. For example, a mark indicating that the third image G3 is a pseudo image (for example, F mark indicating Fake) may be superimposed on the third image G3, or information indicating that the third image G3 is a pseudo image may be written in a header of the third image G3.

The segmentation unit 23 segments the anatomical structure included in the third image G3 derived by the derivation unit 22. The anatomical structure to be segmented varies depending on the imaging part of the subject. For example, in a case in which the imaging part of the third image G3 is a chest, the anatomical structure is segmented as a heart, a lung, a bronchus, and the like. In addition, in a case in which the imaging part of the third image G3 is an abdomen, the anatomical structure is segmented as a liver, a pancreas, and the like.

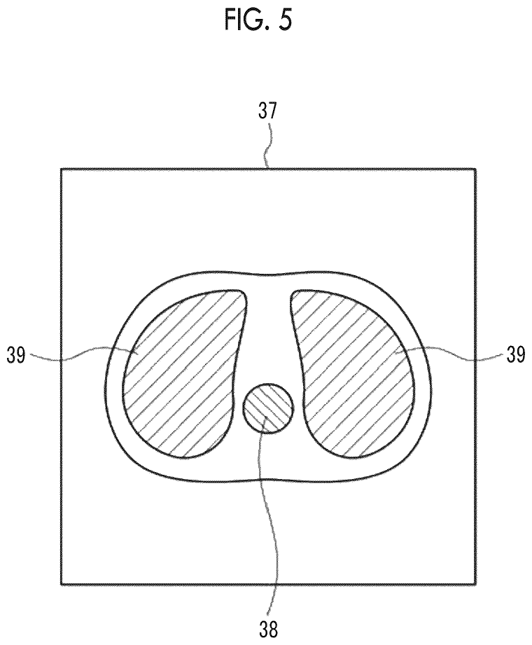

The segmentation unit 23 extracts an anatomical structure from at least one tomographic image included in the third image G3, segments which organ the extracted anatomical structure is, and derives a segmentation result of the anatomical structure, by using the segmentation model 35 that has been trained to segment the anatomical structure included in the image represented by the two-dimensional coordinates. The segmentation result represents the anatomical structure of each pixel in the input image, and the segmentation unit 23 derives a mask representing the segmentation result of the anatomical structure by labeling pixels segmented into the same anatomical structure. FIG. 5 is a diagram showing the mask. A mask 37 shown in FIG. 5 is an axial image of the chest, and a label 38 is assigned to the heart, which is the anatomical structure segmented by the segmentation unit 23, and a label 39 is assigned to the lung.

The mask includes at least any of a mask image indicating a label assigned for each of position coordinates of the pixel, a composite image obtained by combining a modality image generated by a signal value detected by the modality for each of the position coordinates of the pixel and the mask image, or a superimposed image obtained by superimposing the modality image and the mask image. In addition, the segmentation unit 23 may segment the anatomical structure from a target image, which is at least any of the first image G1, the second image G2, or the third image G3, and may derive a segmentation result of the anatomical structure in an image other than the target image based on a correspondence relationship between the target image and the image other than the target image. For example, a mask of the first image G1 may be derived from the first image G1 using the segmentation model 35, and the first image G1 and the mask of the first image G1 may be input to the first conversion model 31 to derive the second image G2 and a mask of the second image G2. In addition, the mask of the second image G2 may be derived from the second image G2 using the segmentation model 35, and the second image G2 and the mask of the second image G2 may be input to the second conversion model 32 to derive the third image G3 and a mask of the third image G3.

The segmentation unit 23 can segment the anatomical structure of not only the MRI image, which is the pseudo image derived by the derivation unit 22, but also the MRI image, which is the actual image acquired by imaging the subject using the MRI apparatus. Therefore, as will be described below, both the actual image and the pseudo image are used to construct the segmentation model 35.

The third image G3 derived by the derivation unit 22 and the mask representing the segmentation result derived by the segmentation unit 23 are associated with each other and transmitted to the image storage server 3 via the network 5 and stored therein.

The learning unit 24 trains the conversion model 30 and the segmentation model 35. First, a case in which only the conversion model 30 is trained will be described. A learning program for causing the CPU 11 to function as the learning unit 24 may be prepared separately from the image processing program 12, and the CPU 11 may be caused to function as the learning unit 24 by the learning program.

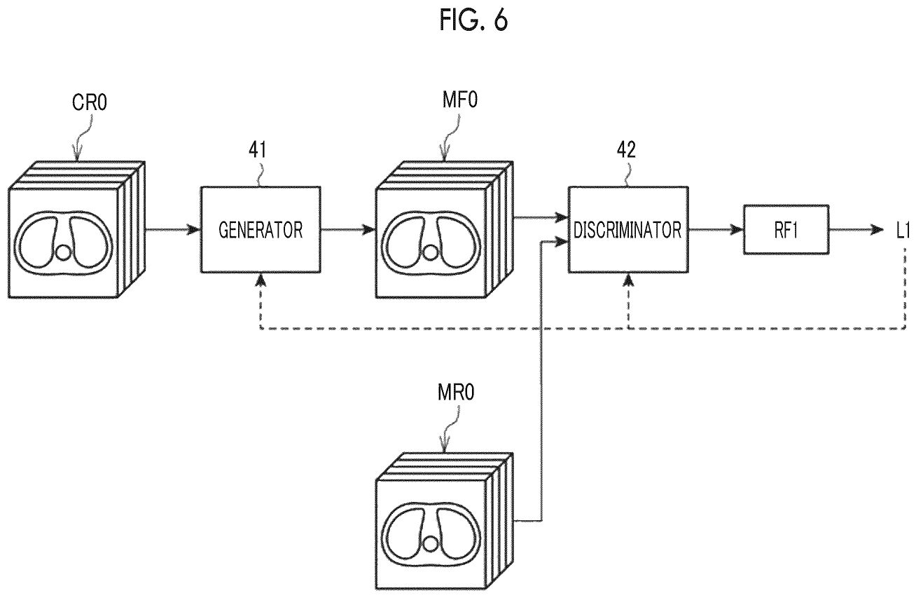

In the present embodiment, the learning unit 24 constructs the conversion model 30 through adversarial learning. In learning, the conversion model 30 constitutes a generative adversarial network (GAN). FIG. 6 is a diagram for describing learning for constructing the first conversion model 31 included in the conversion model 30. As shown in FIG. 6, in order to construct the first conversion model 31, in the present embodiment, a generator 41 and a discriminator 42 are used. In learning, a three-dimensional CT image CR0, which is an actual image, and a three-dimensional MRI image MR0, which is an actual image, are used. The three-dimensional MRI image MR0 is a T1-weighted image. The three-dimensional CT image CR0 and the three-dimensional MRI image MR0 used for learning do not have to be of the same subject.

The generator 41 constructs the first conversion model 31 and is composed of a three-dimensional convolutional neural network (3D-CNN). In a case of training the generator 41, the learning unit 24 inputs the three-dimensional CT image CR0, which is an actual image, to the generator 41 and causes the generator 41 to output a three-dimensional MRI image (in the present embodiment, a T1-weighted image) MF0. The three-dimensional MRI image MF0 output from the generator 41 is not an actual image acquired by imaging the subject using the MRI imaging apparatus, but is a pseudo image.

The three-dimensional CT image CR0 to be input to the generator 41 may be obtained by isotropically adjusting a physical size, performing random data augmentation processing such as posture conversion and enlargement and reduction, and then cutting out a fixed-size region from the processed image.

The discriminator 42 is composed of, for example, a convolutional neural network, discriminates whether the input image is an actual image or a pseudo image, and outputs a discrimination result RF1. In a case in which the discriminator 42 receives the MRI image MR0, which is an actual image, and discriminates that the received image is an actual image, the discrimination result RF1 is a correct answer. In a case in which the discriminator 42 receives the MRI image MR0, which is an actual image, and discriminates that the received image is a pseudo image, that is, the MRI image MF0 derived by the generator 41, the discrimination result RF1 is an incorrect answer. In a case in which the discriminator 42 discriminates that the received pseudo image is an actual image, the discrimination result RF1 is an incorrect answer, and, in a case in which the discriminator 42 discriminates that the received pseudo image is a pseudo image, the discrimination result RF1 is a correct answer.

The learning unit 24 derives a loss L1 based on the discrimination result RF1 output by the discriminator 42. In the present embodiment, the learning unit 24 trains the discriminator 42 to correct the discrimination result RF1 as to whether the input image is an actual image or a pseudo image derived by the generator 41. Specifically, the learning unit 24 trains the discriminator 42 such that the loss L1 is equal to or less than a predetermined threshold value.

In addition, the learning unit 24 derives the three-dimensional MRI image MF0 from the input actual image, that is, from the three-dimensional CT image CR0, and trains the generator 41 such that the discriminator 42 discriminates the discrimination result RF1 as an incorrect answer. Specifically, the learning unit 24 trains the 3D-CNN constituting the generator 41 such that the loss L1 is equal to or less than a predetermined threshold value.

As the learning progresses, the generator 41 and the discriminator 42 improve the accuracy, and the discriminator 42 can more accurately discriminate whether the input MRI image is an actual image or a pseudo image regardless of the type of the input MRI image. Meanwhile, the generator 41 can generate a pseudo image that is not discriminated by the discriminator 42 and that is closer to the MRI image, which is an actual image, from the three-dimensional CT image. By proceeding with the learning in this manner, the generator 41 is constructed as the first conversion model 31.

As the learning for constructing the first conversion model 31, a CycleGAN method disclosed in “Unpaired Image-to-Image Translation using Cycle-Consistent Adversarial Networks, Jun-Yan Zhu, arxiv:1703.10593” may be used. In addition, a three-dimensional CT image and a three-dimensional MRI image for the same part may be prepared, and the generator 41 may perform the learning using a difference between an MRI image derived from a CT image and the prepared MRI image as a loss.

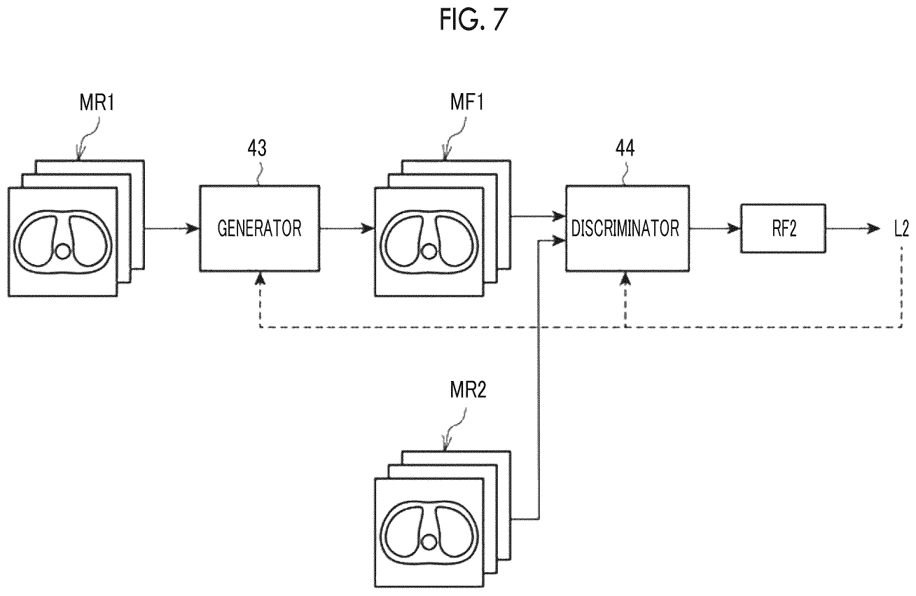

Next, construction of the second conversion model 32 will be described. FIG. 7 is a diagram for describing learning for constructing the second conversion model 32 included in the conversion model 30. As shown in FIG. 7, in order to construct the second conversion model 32, in the present embodiment, a generator 43 and a discriminator 44 are used as with the generator 41 and the discriminator 42 shown in FIG. 6. In learning, one or more MRI-T1-weighted images, which are actual images represented by two-dimensional coordinates, and one or more MRI images, which are other than the MRI-T1-weighted images and are actual images represented by two-dimensional coordinates, are used. Here, it is assumed that a diffusion-weighted image is used as the MRI image other than the MRI-T1-weighted image. The MRI-T1-weighted image and the diffusion-weighted image used for learning do not have to be of the same subject.

The generator 43 is composed of a two-dimensional convolutional neural network (2D-CNN). In a case of training the generator 43, the learning unit 24 inputs an MRI-T1-weighted image MR1, which is an actual image and is represented by two-dimensional coordinates, to the generator 43 and causes the generator 43 to output a diffusion-weighted image MF1 represented by two-dimensional coordinates. In a case in which an MRI-T1-weighted image represented by a plurality of two-dimensional coordinates, that is, a two-dimensional MRI-T1-weighted image is used, the learning unit 24 inputs each of the MRI-T1-weighted images represented by the two-dimensional coordinates to the generator 43 and causes the generator 43 to output the diffusion-weighted image MF1 for each of the input MRI-T1-weighted images. The diffusion-weighted image MF1 output from the generator 43 is not an actual image acquired by imaging the subject using the MRI imaging apparatus, but is a pseudo image.

The MRI-T1-weighted image MR1, which is represented by two-dimensional coordinates, to be input to the generator 43 may be obtained by isotropically adjusting a physical size, performing random data augmentation processing such as posture conversion and enlargement and reduction, and then cutting out a fixed-size region from the processed image.

The discriminator 44 is composed of, for example, a convolutional neural network, discriminates whether the input image is an actual image or a pseudo image, and outputs a discrimination result RF2. In a case in which the discriminator 44 receives a diffusion-weighted image MR2, which is an actual image, and discriminates that the received image is an actual image, the discrimination result RF2 is a correct answer. In a case in which the discriminator 44 receives the diffusion-weighted image MR2, which is an actual image, and discriminates that the received image is a pseudo image, that is, the diffusion-weighted image MF1 derived by the generator 43, the discrimination result RF2 is an incorrect answer. In a case in which the discriminator 44 discriminates that the received pseudo image is an actual image, the discrimination result RF2 is an incorrect answer, and, in a case in which the discriminator 44 discriminates that the received pseudo image is a pseudo image, the discrimination result RF2 is a correct answer.

The learning unit 24 derives a loss L2 based on the discrimination result RF2 output by the discriminator 44. In the present embodiment, the learning unit 24 trains the discriminator 44 to correct the discrimination result RF2 as to whether the input image is an actual image or a pseudo image derived by the generator 43. Specifically, the learning unit 24 trains the discriminator 44 such that the loss L2 is equal to or less than a predetermined threshold value.

In addition, the learning unit 24 derives the diffusion-weighted image MF1 represented by the two-dimensional coordinates from the input actual image, that is, from the MRI-T1-weighted image MR1 represented by the two-dimensional coordinates, and trains the generator 43 such that the discriminator 44 discriminates the discrimination result RF2 as an incorrect answer. Specifically, the learning unit 24 trains the 2D-CNN constituting the generator 43 such that the loss L2 is equal to or less than a predetermined threshold value.

As the learning progresses, the generator 43 and the discriminator 44 improve the accuracy, and the discriminator 44 can more accurately discriminate whether the input MRI image is an actual image or a pseudo image regardless of the type of the input MRI image represented by the two-dimensional coordinates. Meanwhile, the generator 43 can generate a pseudo image that is not discriminated by the discriminator 44 and that is closer to the diffusion-weighted image, which is an actual image, from the MRI-T1-weighted image represented by two-dimensional coordinates. By proceeding with the learning in this manner, the generator 43 is constructed as the second conversion model 32.

In a case in which the second conversion model 32 is constructed to be capable of converting the expression format of the input image into a plurality of types of expression formats, for example, a StarGAN method disclosed in “Yunjey Choi, Minje Choi, Munyoung Kim, Jung-Woo Ha, Sunghun Kim, Jaegul Choo “StarGAN: Unified Generative Adversarial Networks for Multi-Domain Image-to-Image Translation”, arXiv:1711.09020” may be used. In addition, an MUNIT method disclosed in “Xun Huang, Ming-Yu Liu, Serge Belongie, Jan Kautz, Multimodal Unsupervised Image-to-Image Translation, arXiv:1804.04732” may be used.

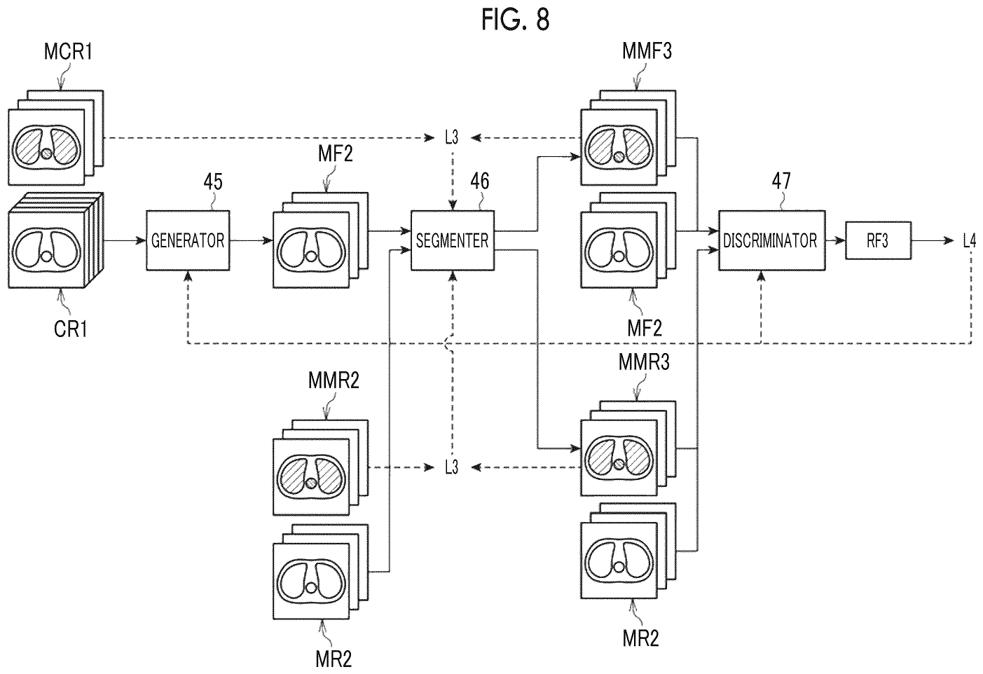

Next, a case in which the conversion model 30 and the segmentation model 35 are trained simultaneously will be described. The learning unit 24 constructs the conversion model 30 and the segmentation model 35 through adversarial learning. In learning, the conversion model 30 and the segmentation model 35 constitute an adversarial generation network. FIG. 8 is a diagram for describing learning for constructing the conversion model 30 and the segmentation model 35. As shown in FIG. 8, in order to construct the conversion model 30 and the segmentation model 35, in the present embodiment, a generator 45, a segmenter 46, and a discriminator 47 are used.

In learning, a three-dimensional CT image CR1 which is an actual image, a mask MCR1 representing a segmentation result of an anatomical structure included in the three-dimensional CT image CR1, a two-dimensional MRI image MR2 which is an actual image, and a mask MMR2 which is a segmentation result of an anatomical structure included in the two-dimensional MRI image MR2 are used. The three-dimensional CT image CR1 and the two-dimensional MRI image MR2 used for learning do not have to be of the same subject.

The generator 45 constructs the conversion model 30 including the first conversion model 31 and the second conversion model 32, and includes the generator 41 shown in FIG. 6 and the generator 43 shown in FIG. 7. In a case of training the generator 45, the learning unit 24 inputs the three-dimensional CT image CR1, which is an actual image, to the generator 45 and causes the generator 45 to output one or more MRI images (T1-weighted image) MF2 represented by two-dimensional coordinates. In a case in which the MRI image MF2 which has a smaller slice interval than the three-dimensional CT image and is represented by a plurality of two-dimensional coordinates or the MRI image MF2 which has a larger slice thickness than the three-dimensional CT image and is represented by one or more two-dimensional coordinates is output, the output MRI image MF2 constitutes the two-dimensional image.

The segmenter 46 is composed of, for example, a two-dimensional convolutional neural network (2D-CNN) and segments the anatomical structure included in the input image. The segmenter 46 performs processing of deriving a probability of being an anatomical structure for each of various anatomical structures included in the input image and segmenting an anatomical structure whose probability is equal to or greater than a threshold value as the anatomical structure. The segmentation result represents the anatomical structure of each pixel in the input image, and a mask representing the segmentation result of the anatomical structure is acquired by labeling pixels segmented into the same anatomical structure.

In addition, both the MRI image MR2, which is an actual image and is represented by two-dimensional coordinates, and the MRI image MF2, which is a pseudo image and is represented by two-dimensional coordinates, are used for training the segmenter 46. Reference numerals of the mask acquired based on the MRI image MR2, which is an actual image, and the mask acquired based on the MRI image MF2, which is a pseudo image, are denoted by MMR3 and MMF3, respectively. In addition, in a case of training the segmenter 46, it is preferable to use the MRI image MR2, which is an actual image, more than the MRI image MF2, which is a pseudo image, in order to improve the training accuracy.

The discriminator 47 is composed of, for example, a convolutional neural network, and, in a case in which an image and a mask of an anatomical structure included in the image are input, the discriminator 47 discriminates whether the mask is acquired by segmenting an actual image or whether the mask is acquired by segmenting a pseudo image, and outputs a discrimination result RF3. In a case in which the discriminator 47 receives the mask MMR3 acquired based on the MRI image MR2, which is an actual image, and the MRI image MR2, and discriminates that the input mask MMR3 is a mask acquired based on an actual image, the discrimination result RF3 is a correct answer. On the other hand, in a case in which the discriminator 47 receives the mask MMR3 acquired based on the MRI image MR2, which is an actual image, and the MRI image MR2, and discriminates that the input mask MMR3 is a mask acquired based on the pseudo image, that is, the MRI image MF2 derived by the generator 45, the discrimination result RF3 is an incorrect answer. In addition, in a case in which the discriminator 47 discriminates that the mask MMF3 acquired based on the input pseudo image (that is, the MRI image MF2) is a mask acquired based on an actual image, the discrimination result RF3 is an incorrect answer, and, in a case in which the discriminator 47 discriminates that the mask MMF3 is a mask acquired based on a pseudo image, the discrimination result RF3 is a correct answer.

The learning unit 24 derives a difference between the mask MMF3 output by the segmenter 46 in response to the input of the MRI image MF2, which is a pseudo image, and the mask MCR1 representing the segmentation result of the anatomical structure included in the CT image CR1, as a loss L3. In addition, a loss between the mask MMR3 output by the segmenter 46 in response to the input of the MRI image MR2, which is an actual image, and the mask MMR2 of the MRI image MR2 is also derived as the loss L3.

The loss L3 can be derived by, for example, Equation (1) or Equation (2). Correct answer data is data of the mask MCR1 or the mask MMR2, and inference data is data of the mask MMF3 or the mask MMR3. Equation (1) is a binary cross-entropy loss, and Equation (2) is a means square error (MSE). In Equations (1) and (2), a left side is the loss L3. In addition, in Equations (1) and (2), in a case in which the loss for the mask MMR3 derived based on the MRI image MR2, which is an actual image, is derived, a weight wi is set to be larger than in a case in which the loss for the mask MMF3 derived based on the MRI image MF2, which is a pseudo image, is derived. As a result, the segmenter 46 can be trained to perform segmentation with higher accuracy.

$$Loss ( y , \hat{y} ) = - \sum {w}_{i} ( {y}_{i} log ( \hat{{y}_{i}} ) + ( 1 - {y}_{i} ) log ( 1 - \hat{{y}_{i}} ) ) \qquad ( 1 )$$

$$Loss ( y , \hat{y} ) = \frac{1}{n} \sum {{w}_{i} ( {y}_{i} - \hat{{y}_{i}} )}^{2} \qquad ( 2 )$$

yᵢ is correct answer data, ŷᵢ is inference data, wᵢ is weight of i-th sample, and n is the number of samples.

In a case of performing the multi-class segmentation, the loss L3 may be derived using Equation (3). In Equation (3), j represents different classes (for example, a heart and a lung), and wij is a weight for a j-th class of an i-th sample.

$$Loss ( y , \hat{y} ) = - \sum \sum {w}_{i j} {y}_{i} log ( \hat{{y}_{i}} ) \qquad ( 3 )$$

The learning unit 24 trains the segmenter 46 such that the loss L3 is equal to or less than a predetermined threshold value.

In addition, the learning unit 24 derives a loss L4 based on the discrimination result RF3 output by the discriminator 47. In the present embodiment, the learning unit 24 trains the discriminator 47 to correct the discrimination result RF3 as to whether the input mask is derived based on an actual image or derived based on a pseudo image derived by the generator 45. Specifically, the learning unit 24 trains the discriminator 47 such that the loss L4 is equal to or less than a predetermined threshold value.

In addition, the learning unit 24 derives the MRI image MF2 represented by two-dimensional coordinates from the input actual image, that is, from the three-dimensional CT image CR1, and trains the generator 45 such that the discriminator 47 discriminates the discrimination result RF3 as an incorrect answer. Specifically, the learning unit 24 trains the 3D-CNN and the 2D-CNN constituting the generator 45 such that the loss L4 is equal to or less than a predetermined threshold value.

As the learning progresses, the generator 45, the segmenter 46, and the discriminator 47 improve the accuracy, and the discriminator 47 can more accurately discriminate whether the input mask is a mask derived based on an actual image or a mask derived based on a pseudo image, regardless of the type of the input mask. Meanwhile, the generator 45 can generate a pseudo image that is not discriminated by the discriminator 47 and that is closer to the MRI image which is an actual image. In addition, in a case in which the MRI image is input to the segmenter 46, whether the MRI image is an actual image or a pseudo image, the segmenter 46 can accurately segment the anatomical structure included in the MRI image. By proceeding with the learning in this manner, the generator 45 is constructed as the conversion model 30, and the segmenter 46 is constructed as the segmentation model 35.

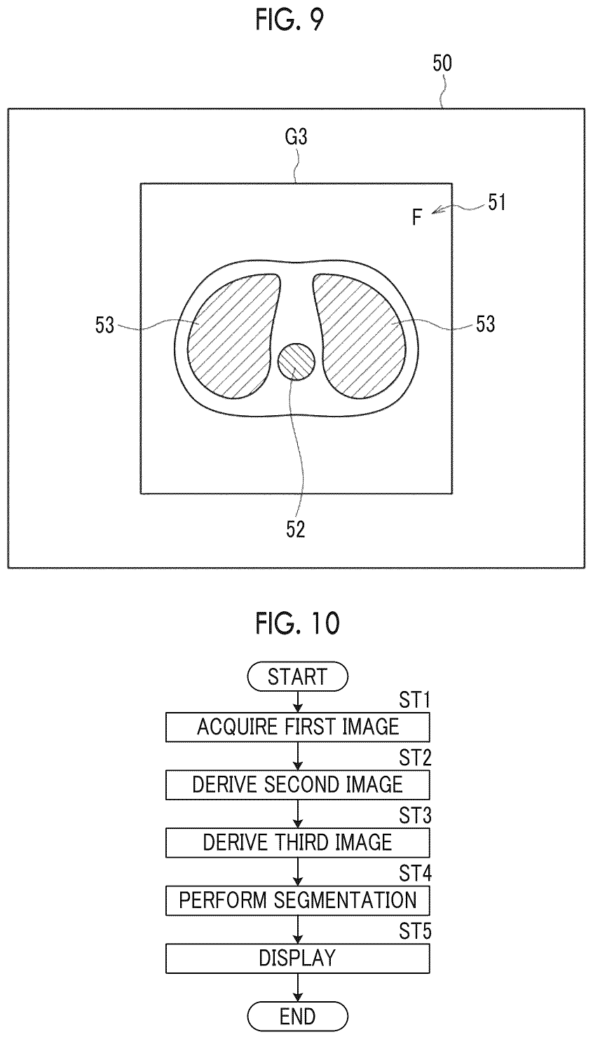

The display control unit 25 displays the derived third image G3. FIG. 9 is a diagram showing a display screen of the third image. As shown in FIG. 9, the third image G3, that is, the MRI image is displayed on a display screen 50. A mark 51 of “F”, which is information indicating that the third image G3 is a pseudo image, is displayed simultaneously with the third image G3. In addition, masks 52 and 53 are assigned to the heart and the lung included in the third image G3, respectively. As a result, a person who views the third image G3 can recognize that the third image G3 is derived in a pseudo manner. In a case in which there are a plurality of the third images G3, the plurality of third images G3 can be switched and displayed in response to an instruction from the input device 15.

Next, processing performed in the present embodiment will be described. FIG. 10 is a flowchart showing the processing performed in the present embodiment. First, the information acquisition unit 21 acquires the first image G1 from the image storage server 3 (step ST1). Next, the derivation unit 22 derives the second image G2 from the first image G1 of the first modality using the first conversion model 31 (step ST2), and derives the third image G3 of the second modality different from the first modality from the second image G2 using the second conversion model 32 (step ST3). Next, the segmentation unit 23 segments the anatomical structures included in the third image G3 (step ST4). Then, the display control unit 25 displays the third image G3 on the display 14 (step ST5), and the processing is ended.

As described above, in the present embodiment, the second image G2 is derived from the first image G1 of the first modality using the first conversion model 31, and the third image G3 of the second modality different from the first modality is derived from the second image G2 using the second conversion model 32. Therefore, a gap in expression format, that is, a domain gap in each of a case of performing conversion from the first image G1 into the second image G2 and a case of performing conversion from the second image G2 into the third image G3 is smaller than in a case of performing direct conversion from the first image G1 into the third image G3. Therefore, in the conversion from the first image G1 into the second image G2 and the conversion from the second image G2 into the third image G3, it is possible to prevent the anatomical structure in the image from being destroyed, and as a result, it is possible to perform the domain conversion of the image in which the anatomical structure is maintained.

Next, the learning device according to the present embodiment will be described. The learning device 4 according to the present embodiment constructs a segmentation model for segmenting an anatomical structure included in an image. Specifically, a segmentation model for segmenting an anatomical structure included in an MRI image is constructed.

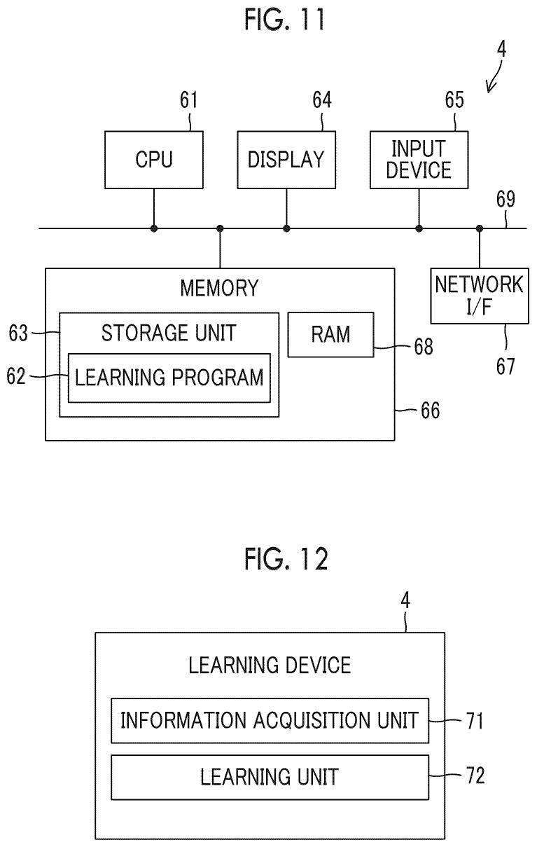

FIG. 11 is a diagram showing a hardware configuration of the learning device according to the present embodiment. As shown in FIG. 11, the learning device 4 includes a CPU 61, a display 64, an input device 65, a memory 66, and a network I/F 67 connected to a network. The CPU 61, the display 64, the input device 65, the memory 66, and the network I/F 67 are connected to a bus 69. The memory 66 includes a storage unit 63 and a RAM 68. The CPU 61 is an example of a processor in the present disclosure. The CPU 61, the display 64, the input device 65, the memory 66, and the network I/F 67 correspond to the CPU 11, the display 14, the input device 15, the memory 16, and the network I/F 17 shown in FIG. 2, so that detailed description thereof will be omitted here. The storage unit 63 stores a learning program 62 according to the present embodiment.

FIG. 12 is a diagram showing a functional configuration of the learning device according to the present embodiment. As shown in FIG. 12, the learning device 4 according to the present embodiment comprises an information acquisition unit 71 and a learning unit 72. In a case in which the CPU 61 executes the learning program 62, the CPU 61 functions as the information acquisition unit 71 and the learning unit 72.

The information acquisition unit 71 acquires learning data for use in learning from the image storage server 3 via the network 5. The learning data used in the present embodiment is a pair formed of the third image G3 derived by the image processing apparatus 1 according to the present embodiment and a mask acquired based on the third image G3, and a pair formed of an actual image in the same expression format as the third image G3 and a mask derived based on the actual image. The third image G3 and the actual image used for learning include a two-dimensional image, that is, at least one image represented by two-dimensional coordinates in which a slice interval or a slice thickness is larger than that of a three-dimensional image.

The learning device 4 according to the present embodiment trains a segmenter for constructing a segmentation model for segmenting the anatomical structure included in the MRI image as described above. Therefore, the information acquisition unit 71 acquires, as first learning data 81, a pair formed of an MRI image MR5, which is an actual image acquired by imaging the subject using the MRI apparatus, and a mask MMR5 representing a segmentation result of the anatomical structure included in the MRI image MR5. In addition, the information acquisition unit 71 acquires, as second learning data 82, a pair formed of an MRI image MF5, which is the third image G3 derived by the image processing apparatus 1 according to the present embodiment, and a mask MMF5 representing a segmentation result of the anatomical structure included in the MRI image MF0.

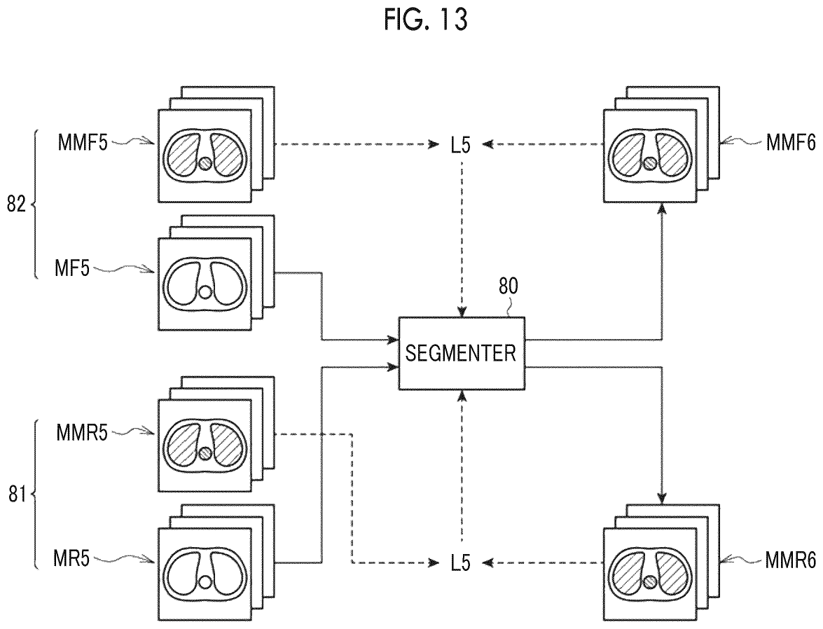

The learning unit 72 trains a segmenter in order to construct a segmentation model for segmenting the anatomical structure included in the image. FIG. 13 is a diagram for describing training of the segmenter for constructing the segmentation model. The segmenter 80 is composed of, for example, a two-dimensional convolutional neural network (2D-CNN) and segments the anatomical structure included in the input image represented by two-dimensional coordinates. The first learning data 81 and the second learning data 82 acquired by the information acquisition unit 71 are used for learning.

In this case, the learning unit 72 inputs the MRI image MR5 represented by the two-dimensional coordinates included in the first learning data 81 to the segmenter 80, and derives a mask MMR6 representing a segmentation result of the anatomical structure. In addition, the learning unit 72 inputs the MRI image MF5 represented by the two-dimensional coordinates included in the second learning data 82 to the segmenter 80, and derives a mask MMF6 representing a segmentation result of the anatomical structure. Then, the learning unit 72 derives a difference between the mask MMR5 and the mask MMR6 and a difference between the mask MMF5 and the mask MMF6 as a loss L5, and trains the segmenter 80 such that the loss L5 is equal to or less than a predetermined threshold value, thereby constructing a segmentation model.

In training the segmenter 80, the number of samples of the first learning data 81 may be greater than the number of samples of the second learning data 82. In addition, in a case of deriving the loss L5, as in a case in which the segmentation model 35 in the image processing apparatus 1 according to the present embodiment is constructed, the weighting of the loss for the first learning data 81 may be increased.

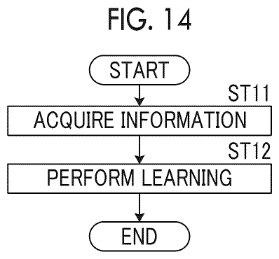

Next, processing performed by the learning device 4 according to the present embodiment will be described. FIG. 14 is a flowchart showing the processing performed by the learning device according to the present embodiment. First, the information acquisition unit 71 acquires the first learning data and the second learning data from the image storage server 3 (learning data acquisition: step ST11). Next, the learning unit 72 constructs a segmentation model by training the segmenter using the first learning data and the second learning data (step ST12), and the processing is ended.

As described above, in the learning device according to the present embodiment, the third image G3 derived by the image processing apparatus 1 according to the present embodiment is used as the learning data. Therefore, the amount of the learning data can be increased, so that a segmentation model for segmenting the anatomical structure included in the image can be constructed with high accuracy.

In the embodiment of the learning device, the first learning data and the second learning data are used for training the segmenter, but the present invention is not limited to this. The segmenter may be trained using only the second learning data including the third image G3 derived by the image processing apparatus 1 according to the present embodiment.

In addition, in the embodiment of the image processing apparatus, the first conversion model 31 converts the first image G1 (three-dimensional CT image) into the second image G2 (three-dimensional MRI-T1-weighted image) having a different modality, the two-dimensional image is extracted from the second image G2, and the second conversion model 32 converts the two-dimensional second image G2 into the third image G3 having the same modality but a different expression format, but the present invention is not limited to this. The second conversion model 32 may convert the two-dimensional second image G2 into a two-dimensional third image G3 having a modality different from the modality of the second image G2. That is, the first image G1 corresponds to the first modality, the second image G2 corresponds to the second modality, the third image G3 corresponds to the third modality, and the third modality may be different from the first modality and the second modality. In this case, for example, the second conversion model 32 need only be constructed to convert the two-dimensional MRI image into an X-ray image, an ultrasound image, a PET image, or the like other than MRI.

In addition, the first conversion model 31 may convert the three-dimensional first image G1 into a three-dimensional second image G2 having the same modality but a different expression format. In this case, the second conversion model 32 need only convert the second image G2, which is a two-dimensional image extracted from the three-dimensional second image G2, into a two-dimensional third image G3 having a modality different from the modality of the first image G1. Examples of the images having the same modality but different expression formats include CT contrast and non-contrast images, images having different kernels such as a lung field and a mediastinum, images having at least one of a slice interval or a slice thickness different from each other, and different types of MRI images. In this case, for example, the first conversion model 31 need only be constructed to convert a three-dimensional CT contrast image into a three-dimensional CT non-contrast image, and the second conversion model 32 need only be constructed to convert a two-dimensional CT non-contrast image extracted from the three-dimensional CT non-contrast image into a two-dimensional MRI-T1-weighted image.

In addition, the conversion model 30 may derive the three-dimensional third image G3 from the two-dimensional first image G1. In this case, the first conversion model 31 converts a plurality of two-dimensional first images G1 into a plurality of two-dimensional second images G2 having modalities different from the modality of the first image G1, and derives a three-dimensional second image G2 by performing slice interpolation or the like on the two-dimensional second images G2. The second conversion model 32 may convert the three-dimensional second image G2 into a three-dimensional third image G3 in an expression format different from the expression format of the second image G2. In this case, for example, the first conversion model 31 need only be constructed to convert a two-dimensional CT image into a two-dimensional MRI-T2-weighted image, and the second conversion model 32 need only be constructed to convert a three-dimensional MRI-T2-weighted image into a three-dimensional MRI-T1-weighted image. For example, the first conversion model 31 is composed of a 2D-CNN, and the second conversion model 32 is composed of a 3D-CNN.

In addition, the first conversion model 31 may convert a plurality of two-dimensional first images G1 into a plurality of two-dimensional second images G2 having modalities different from the modality of the first image G1, and derive a three-dimensional second image G2 from the two-dimensional second image G2, and the second conversion model 32 may convert the three-dimensional second image G2 into a three-dimensional third image G3 having a modality different from the modality of the second image G2. In this case, for example, the first conversion model 31 need only be constructed to convert a two-dimensional CT image into a two-dimensional MRI-T2-weighted image, and the second conversion model 32 need only be constructed to convert a three-dimensional MRI-T2-weighted image into a three-dimensional PET image. For example, the first conversion model 31 is composed of a 2D-CNN, and the second conversion model 32 is composed of a 3D-CNN.

In addition, the first conversion model 31 may convert the two-dimensional first image G1 into a two-dimensional second image G2 having the same modality but a different expression format. In this case, the three-dimensional second image G2 may be derived from the two-dimensional second image G2, and the second conversion model 32 need only convert the three-dimensional second image G2 may be converted into the three-dimensional third image G3 having a modality different from the first image G1. Examples of the images having the same modality but different expression formats include CT contrast and non-contrast images, images having different kernels such as a lung field and a mediastinum, images having at least one of a slice interval or a slice thickness different from each other, and different types of MRI images. In this case, for example, the first conversion model 31 may be constructed to convert a two-dimensional CT contrast image into a two-dimensional CT non-contrast image, and the second conversion model 32 may be constructed to convert a three-dimensional CT non-contrast image extracted from the two-dimensional CT non-contrast image into a three-dimensional MRI-T1-weighted image. For example, the first conversion model 31 is composed of a 2D-CNN, and the second conversion model 32 is composed of a 3D-CNN.

In addition, in the above-described embodiment, the image processing apparatus 1 includes the learning unit 24, but the present invention is not limited to this. The conversion model 30 and the segmentation model 35 may be constructed by a learning device separate from the image processing apparatus 1, and the constructed conversion model 30 and segmentation model 35 may be applied to the derivation unit 22 and the segmentation unit 23 of the image processing apparatus 1.

In this embodiment, each process is executed on an arbitrary computer. The arbitrary computer may execute these processes by means of a processor as hardware, a program as software, or a combination of the processor and the program. In such a case, the processor is configured to execute the various processes in this embodiment in cooperation with the program and may function as each unit or means in this embodiment. In addition, the order in which the processor executes these processes is not limited to the order described in this embodiment and may be changed as appropriate. The arbitrary computer may be a general-purpose computer, a computer for a specific purpose, a workstation, or any other system capable of executing each process.

The processor may be configured by one or more hardware, and the type of hardware is not limited. For example, the processor may comprise at least one of programmable logic devices such as CPUs (Central Processing Units), MPUs (Micro Processing Units), and FPGAs (Field Programmable Gate Arrays); dedicated circuits for performing specific processes such as ASICs (Application Specific Integrated Circuits); and other hardware such as a GPU (Graphics Processing Unit) and an NPU (Neural Processing Unit). The hardware may also be a combination of different types of hardware. When multiple hardware are configured to execute one or more processes of a processor, the said multiple hardware may exist in devices that are physically separate from each other, or in the same device. In any embodiment, the order of each process by the processor is not limited to the order described above and may be changed as appropriate. The hardware is configured by an electric circuit (circuitry) etc. that combines circuit elements such as semiconductor devices.

Furthermore, the program may be firmware or software such as microcode. The program may also be a group of program modules, each function of which may be performed by a processor configured to execute each of the program modules. The program may be program code or code segments stored on one or more non-transitory computer-readable media (e.g., storage media or other storage). The program may be stored in separate non-transitory computer-readable media located on devices that are physically separate from each other. The program code or code segments may represent any combination of procedures, functions, subprograms, routines, subroutines, modules, software packages, classes, instructions, data structures, or program statements. The program code or code segments may be connected to other code segments or hardware circuits by sending or receiving information, data, arguments, parameters, or memory contents.

In the above embodiment, it is explained that the image processing program 12 is stored (installed) in advance in the storage unit 13, and a learning program 62 is stored (installed) in advance in the storage unit 63, but this is not limited to this. The image processing program 12 and the learning program 62 may be provided in a form recorded on a recording medium such as a CD-ROM (Compact Disc Read Only Memory), DVD-ROM (Digital Versatile Disc Read Only Memory), and USB (Universal Serial Bus) memory. In addition, the image processing program 12 and the learning program 62 may be provided in a form that the image processing program 12 and the learning program 62 are downloaded from an external device via a network.

The technology of this disclosure also extends to all types of program products. Program products include all types of products for providing programs. For example, program products include programs provided via networks such as the Internet, and non-temporary computer readable storage media such as CD-ROMs, DVDs, and USB memory devices that store programs. [0100] Appendices of the present disclosure will be described below.

### APPENDIX 1

An image processing apparatus comprising:
a processor,
in which the processor
   derives a second image from a first image of a first modality, and
   derives a third image of a second modality different from the first modality, from the second image.

### APPENDIX 2

The image processing apparatus according to Appendix 1,
in which the processor
   derives the second image from the first image of the first modality using a first conversion model, and
   derives the third image of the second modality from the second image using a second conversion model.

### APPENDIX 3

The image processing apparatus according to Appendix 2,
in which the first conversion model is a three-dimensional image conversion model, and
the processor derives a three-dimensional second image from a three-dimensional first image using the first conversion model.

### APPENDIX 4

The image processing apparatus according to Appendix 2 or 3,
in which the second conversion model is a two-dimensional image conversion model, and
the processor
   extracts at least one tomographic image from a three-dimensional second image, and
   derives the third image from the tomographic image using the second conversion model.

### APPENDIX 5

The image processing apparatus according to Appendix 2,
in which the first conversion model is a two-dimensional image conversion model, and
the processor derives the second image represented by two-dimensional coordinates from the first image represented by two-dimensional coordinates using the first conversion model.

### APPENDIX 6

The image processing apparatus according to Appendix 2 or 5,
in which the second conversion model is a three-dimensional image conversion model, and
the processor
   derives a three-dimensional second image from the second image represented by a plurality of two-dimensional coordinates, and
   derives the third image from the three-dimensional second image using the second conversion model.

### APPENDIX 7

The image processing apparatus according to Appendix 1,
in which the processor derives a segmentation result for an anatomical structure included in the third image by segmenting the anatomical structure.

### APPENDIX 8

The image processing apparatus according to any one of Appendices 2 to 6,
in which the processor derives a segmentation result for an anatomical structure included in the third image by segmenting the anatomical structure.

### APPENDIX 9

The image processing apparatus according to Appendix 8,
in which the processor
   introduces adversarial learning to a discriminator that discriminates between the segmentation result of the anatomical structure using the third image and a segmentation result of the anatomical structure using an actual image of the same modality as the third image, and
   trains the first conversion model and the second conversion model so as to derive the third image such that the discriminator is unable to discriminate the segmentation result of the anatomical structure for the third image.

### APPENDIX 10

The image processing apparatus according to any one of Appendices 1 to 9,
in which the second image has an expression format different from expression formats of the first image and the third image.

### APPENDIX 11

The image processing apparatus according to any one of Appendices 1 to 10,
in which the processor stores or displays the third image in a manner that allows the third image to be recognized as having been derived by the processor.

### APPENDIX 12

A learning device that performs learning for constructing a segmentation model for segmenting an anatomical structure included in an image of a second modality, the learning device comprising:
a processor,
in which the processor performs the learning using the third image derived by the image processing apparatus according to any one of Appendices 1 to 11 as learning data.

### APPENDIX 13

The learning device according to Appendix 12,
in which the processor performs the learning using a segmentation result of the anatomical structure in the first image from which the third image is derived.

### APPENDIX 14

The learning device according to Appendix 12 or 13,
in which the processor further uses an actual image acquired by the second modality as the learning data to perform the learning.

### APPENDIX 15

The learning device according to Appendix 14,
in which the processor uses the actual image in the learning more frequently than the third image.

### APPENDIX 16

The learning device according to Appendix 14 or 15,
in which the processor weights the actual image more heavily than the third image in a case in which the actual image and the third image are used in the learning.

### APPENDIX 17

An image processing method comprising:
causing a computer to execute
   deriving a second image from a first image of a first modality, and
   deriving a third image of a second modality different from the first modality, from the second image.

### APPENDIX 18

A learning method for performing learning for constructing a segmentation model for segmenting an anatomical structure included in an image of a second modality via a computer,
in which the learning is performed using the third image derived by the image processing apparatus according to any one of Appendices 1 to 11 as learning data.

### APPENDIX 19

An image processing program causing a computer to execute:
a procedure of deriving a second image from a first image of a first modality; and
a procedure of deriving a third image of a second modality different from the first modality, from the second image.

### APPENDIX 20

A learning program causing a computer to execute learning for constructing a segmentation model for segmenting an anatomical structure included in an image of a second modality, the learning program causing the computer to execute the learning using the third image derived by the image processing apparatus according to any one of Appendices 1 to 11 as learning data.

What is claimed is:

1. An image processing apparatus comprising:
a processor,
wherein the processor
derives a second image from a first image of a first modality, and
derives a third image of a second modality different from the first modality, from the second image.

2. The image processing apparatus according to claim 1,
wherein the processor
derives the second image from the first image of the first modality using a first conversion model, and
derives the third image of the second modality from the second image using a second conversion model.

3. The image processing apparatus according to claim 2,
wherein the first conversion model is a three-dimensional image conversion model, and
the processor derives a three-dimensional second image from a three-dimensional first image using the first conversion model.

4. The image processing apparatus according to claim 2,
wherein the second conversion model is a two-dimensional image conversion model, and
the processor
extracts at least one tomographic image from a three-dimensional second image, and
derives the third image from the tomographic image using the second conversion model.

5. The image processing apparatus according to claim 2,
wherein the first conversion model is a two-dimensional image conversion model, and
the processor derives the second image represented by two-dimensional coordinates from the first image represented by two-dimensional coordinates using the first conversion model.

6. The image processing apparatus according to claim 2,
wherein the second conversion model is a three-dimensional image conversion model, and
the processor
derives a three-dimensional second image from the second image represented by a plurality of two-dimensional coordinates, and
derives the third image from the three-dimensional second image using the second conversion model.

7. The image processing apparatus according to claim 1,
wherein the processor derives a segmentation result for an anatomical structure included in the third image by segmenting the anatomical structure.

8. The image processing apparatus according to claim 2,
wherein the processor derives a segmentation result for an anatomical structure included in the third image by segmenting the anatomical structure.

9. The image processing apparatus according to claim 8,
wherein the processor
introduces adversarial learning to a discriminator that discriminates between the segmentation result of the anatomical structure using the third image and a segmentation result of the anatomical structure using an actual image of the same modality as the third image, and
trains the first conversion model and the second conversion model so as to derive the third image such that the discriminator is unable to discriminate the segmentation result of the anatomical structure for the third image.

10. The image processing apparatus according to claim 1,
wherein the second image has an expression format different from expression formats of the first image and the third image.

11. The image processing apparatus according to claim 1,
wherein the processor stores or displays the third image in a manner that allows the third image to be recognized as having been derived by the processor.

12. A learning device that performs learning for constructing a segmentation model for segmenting an anatomical structure included in an image of a second modality, the learning device comprising:
a processor,
wherein the processor performs the learning using the third image derived by the image processing apparatus according to claim 1 as learning data.

13. The learning device according to claim 12,
wherein the processor performs the learning using a segmentation result of the anatomical structure in the first image from which the third image is derived.

14. The learning device according to claim 12,
wherein the processor further uses an actual image acquired by the second modality as the learning data to perform the learning.

15. The learning device according to claim 14,
wherein the processor uses the actual image in the learning more frequently than the third image.

16. The learning device according to claim 14,
wherein the processor weights the actual image more heavily than the third image in a case in which the actual image and the third image are used in the learning.

17. An image processing method comprising:
causing a computer to execute
deriving a second image from a first image of a first modality, and
deriving a third image of a second modality different from the first modality, from the second image.

18. A learning method for performing learning for constructing a segmentation model for segmenting an anatomical structure included in an image of a second modality via a computer,
wherein the learning is performed using the third image derived by the image processing apparatus according to claim 1 as learning data.

19. A non-transitory computer-readable storage medium that stores an image processing program causing a computer to execute:
a procedure of deriving a second image from a first image of a first modality; and
a procedure of deriving a third image of a second modality different from the first modality, from the second image.

20. A non-transitory computer-readable storage medium that stores a learning program causing a computer to execute learning for constructing a segmentation model for segmenting an anatomical structure included in an image of a second modality, the learning program causing the computer to execute the learning using the third image derived by the image processing apparatus according to claim 1 as learning data.
